(19) United States
(12) Patent Application Publication
(10) Pub. No.: US 2012/0083486 A1
(43) Pub. Date: Apr. 5, 2012

(54) KIT, COMPOSITION, PRODUCT OR MEDICAMENT FOR TREATING COGNITIVE IMPAIRMENT

(75) Inventors: Yoshimasa YAMAGUCHI, Tokyo (JP); Toshiyuki Matsuno, Tokyo (JP); Kenichi Saitoh, Tokyo (JP)

(73) Assignee: ZENYAKU KOGYO KABUSHIKIKAISHA, Tokyo (JP)

(21) Appl. No.: 13/326,535

(22) Filed: Dec. 15, 2011

Related U.S. Application Data
(63) Continuation of application No. 12/919,651, filed on Nov. 19, 2010.
(60) Provisional application No. 61/275,694, filed on Feb. 28, 2008.

(51) Int. Cl. A61K 31/55; A61P 25/00; A61P 25/28; A61K 31/437

(52) U.S. Cl. 514/215; 514/278

(57) ABSTRACT

A kit, composition, product or medicament for treating cognitive impairment is provided, which includes a therapeutic agent for neurodegenerative disease and a heterocyclic compound represented by the following General Formula (I): [symbol] or a hydrate thereof, a solvate thereof or a pharmaceutically acceptable salt thereof.

### CROSS-REFERENCE TO RELATED APPLICATIONS

This application is a continuation of U.S. application Ser. No. 12/919,651 filed Nov. 19, 2010, pending, which is a 371 of PCT/JP09/000,918 filed Dec. 27, 2009 and claims the benefit of U.S. 61/275,694 filed Feb. 28, 2008, the content of which is incorporated herein by reference.

### FIELD OF THE INVENTION

The present invention relates to a method for treating cognitive impairment by combining a therapeutic agent for neurodegenerative disease and heterocyclic compounds of specific structures.

### BACKGROUND OF THE INVENTION

In recent years, concomitant therapy in which a plurality of drugs with different functional mechanisms are administered in combination has been used in the drug therapy of many diseases, for the purpose of preventing and treating diseases, slowing the onset of symptoms, complementing or enhancing activity, reducing side effects by reducing the dosage of drugs administered, improving the compliance of patients and suppressing the development of drug resistance.

Alzheimer's disease (AD) is a progressive neurodegenerative disease with cognitive impairment as its main symptom. Under present social conditions in which society is progressively aging, the treatment of cognitive impairment is becoming a very important issue. While four drugs, donepezil hydrochloride, rivastigmine tartrate, galantamine hydrobromide and memantine hydrochloride, are currently recognized as agents for the treatment of AD, only donepezil is currently approved for use in Japan.

Concomitant therapy using drugs with different functional mechanisms as mentioned above has been attempted with a view to making effective use of these few drugs, or to make the transfer from palliative therapy to radical therapy. For example, the effects of the conjunctive use of the acetylcholinesterase inhibitor, donepezil and the NMDA (N-methyl-D-aspartate) inhibitor, memantine have been recognized (JAMA 2004; 291:317-324). Additionally, while still in the stage of development, there have been reports of conjunctive use with FK960 (Pharmacology, Biochemistry and Behavior, 73, 511-519 (2002)).

Cognitive impairment is caused not only by AD, but also by various other conditions such as cerebrovascular disease, Lewy body dementia and Parkinson's disease. Therefore, it is important to look for a wide range of drugs with concomitant effects for such cognitive impairments. On the other hand, cognitive enhancers containing heterocyclic compounds with imidazo[1,2-a]pyridin-2(3H)-one within their basic skeletal structures are disclosed in WO 01/09131 and WO 02/060907.

However, these heterocyclic compounds are disclosed as cognitive enhancers for treating memory impairment and memory acquisition/storage impairment in sufferers of AD and senile dementia, and there is no disclosure of activity relating to concomitant use with existing therapeutic agents for neurodegenerative disease. Additionally, these heterocyclic compounds have been found to have different functional mechanisms from existing drugs, due to the fact that they do not have an acetylcholinesterase inhibiting function, but rather increase the amount of free acetylcholine and dopamine (Neurosci. Res. 2002, 26 (suppl): 5131; J. Pharmacol. Exp. Ther. 317:1079-1087 (2006)).

### [Patent Document 1]

WO 01/09131

[Patent Document 2]
WO 02/060907

### [Non Patent Document 1]

JAMA 2004; 291:317-324

### [Non Patent Document 2]

Pharmacology, Biochemistry and Behavior, 73, 511-519 (2002)

### [Non Patent Document 3]

Neurosci. Res. 2002, 26 (suppl): 5131

### [Non Patent Document 4]

J. Pharmacol. Exp. Ther. 317:1079-1087 (2006)

### SUMMARY OF INVENTION

The present invention provides a kit or medicament for treating cognitive impairment, including a therapeutic agent for neurodegenerative disease and a heterocyclic compound represented by the following General Formula (I):

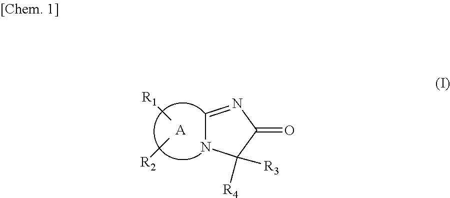

or a hydrate thereof, a solvate thereof or a pharmaceutically acceptable salt thereof.

The present invention also provides a pharmaceutical composition for treating cognitive impairment, including a therapeutic agent for neurodegenerative disease and a heterocyclic compound represented by the following General Formula (I):

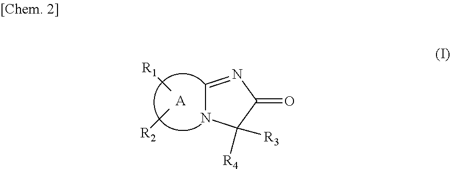

or a hydrate thereof, a solvate thereof or a pharmaceutically acceptable salt thereof.

The present invention also provides a composition including a therapeutic agent for neurodegenerative disease and a heterocyclic compound represented by the following General Formula (I):

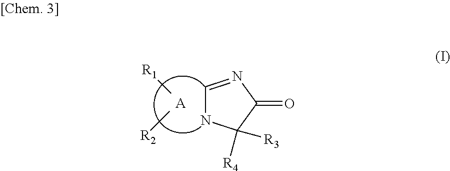

or a hydrate thereof, a solvate thereof or a pharmaceutically acceptable salt thereof.

The present invention also provides a product including a therapeutic agent for neurodegenerative disease and a heterocyclic compound represented by the following General Formula (I):

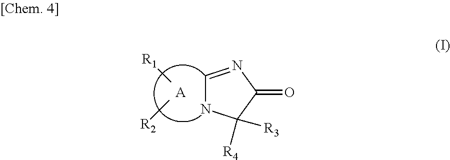

or a hydrate thereof, a solvate thereof or a pharmaceutically acceptable salt thereof, as a combined preparation for simultaneous, separate or sequential use in treatment of cognitive impairment.

The present invention also provides a medicament including a therapeutic agent for neurodegenerative disease, for use in treatment of cognitive impairment in combination with a heterocyclic compound represented by the following General Formula (I):

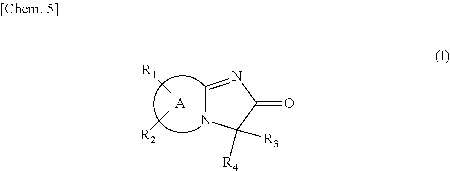

or a hydrate thereof, a solvate thereof or a pharmaceutically acceptable salt thereof.

The present invention also provides a medicament including a heterocyclic compound represented by the following General Formula (I):

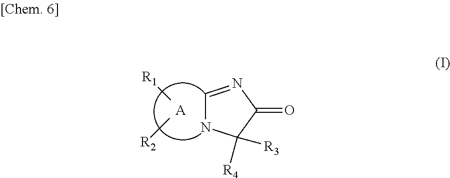

or a hydrate thereof, a solvate thereof or a pharmaceutically acceptable salt thereof, for use in treatment of cognitive impairment in combination with a therapeutic agent for neurodegenerative disease.

The present invention provides a method for treating cognitive impairment by means of a therapeutic agent for neurodegenerative disease and a heterocyclic compound represented by the following General Formula (I):

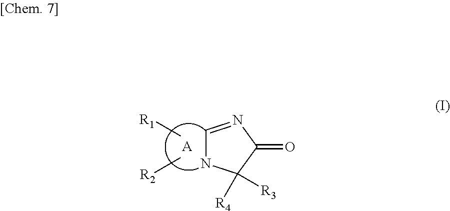

or a hydrate thereof, a solvate thereof or a pharmaceutically acceptable salt thereof.

In the General Formula (I), the structural unit having the General Formula (II):

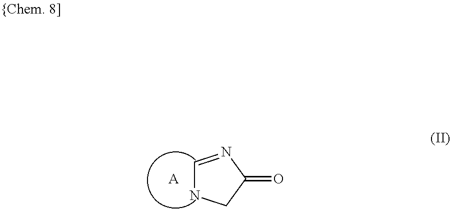

is selected from multiple types of structural units having the General Formula (III):

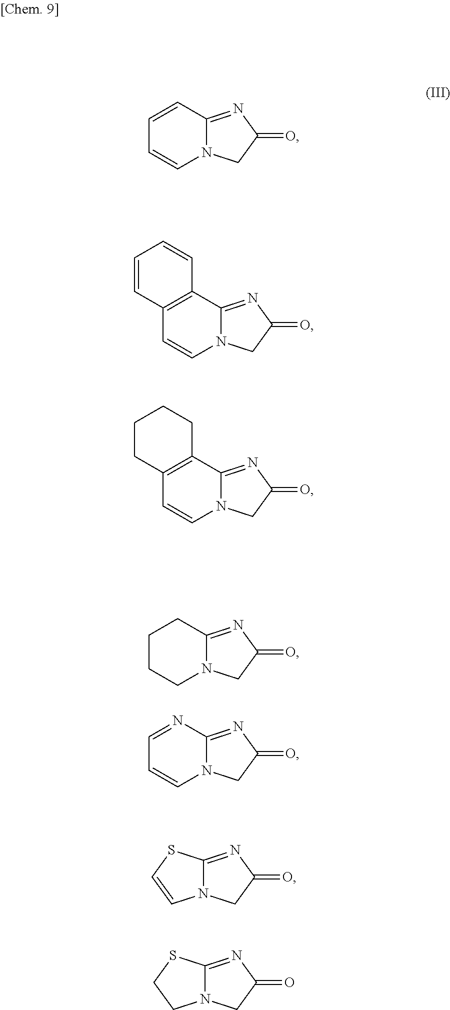

Furthermore, in the General Formula (I), R₁ and R₂ each are independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, and —O(CH₂)ₙ—R₅, wherein R₅ is a vinyl group, C₃-C₅ cycloalkyl group, or phenyl group, and n is 0 or 1.

Furthermore, in the General Formula (I), R₃ and R₄ each are independently selected from the group consisting of a hydrogen atom, C₁-C₆ alkyl group, C₃-C₈ cycloalkyl group, and —CH(R₇)—R₆; alternatively, R₃ and R₄ together form a spiro ring having the General Formula (IV):

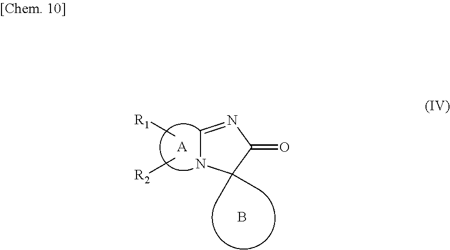

The above R₅ is selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group. The above R₇ is a hydrogen atom or C₁-C₆ alkyl group.

In the General Formula (IV), the structural unit B is selected from multiple types of structural units having the General Formula (V). The structural unit B binds at a position marked by * in the General Formula (V) to form a spiro ring:

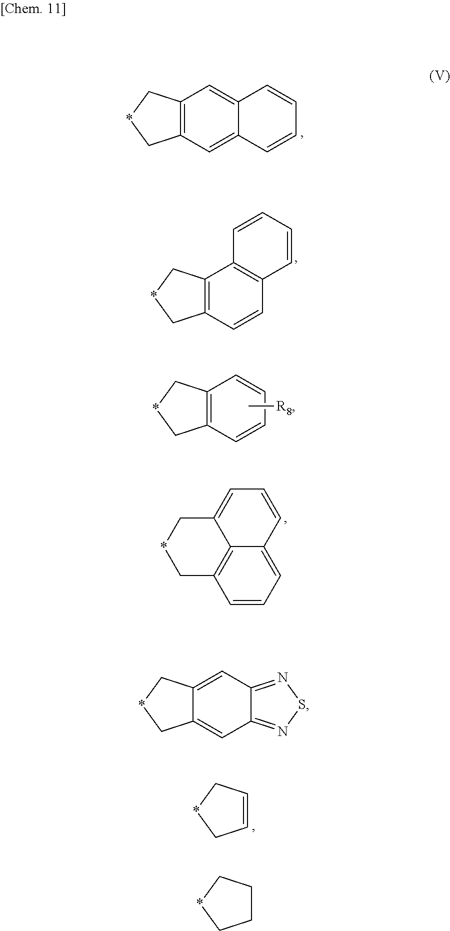

Here, R₅ is one or more substituent group(s) selected from the group consisting of a hydrogen atom, halogen atom, hydroxy group, C₁-C₆ alkoxy group, cyano group, and trifluoromethyl group.

The heterocyclic compound is preferably at least one heterocyclic compound chosen from the group consisting of:
spiro[imidazo[1,2-a]pyridin-2(3H)-one-3,2′-indan],
3,3-dibenzyl-8-isopropoxyimidazo[1,2-a]pyridin-2(3H)-one,
3,3-dibenzyl-8-methoxyimidazo[1,2-a]pyridin-2(3H)-one,
3,3-dibenzyl-8-cyclopropylmethyloxy-imidazo[1,2-a]pyridin-2(3H)-one,
3,3-dibenzyl-6-chloroimidazo[1,2-a]pyridin-2(3H)-one,
8-allkyloxy-3,3-dibenzylimidazo[1,2-a]pyridin-2(3H)-one,
3,3-dibenzyl-8-benzyloxyimidazo[1,2-a]pyridin-2(3H)-one,
8-benzyloxy-3,3-bis(1-phenylethyl)imidazo[1,2-a]pyridin-2(3H)-one,
3,3-dibenzyl-8-methylimidazo[1,2-a]pyridin-2(3H)-one,
3,3-dibenzyl-5,7-dimethylimidazo[1,2-a]pyridin-2(3H)-one,
3,3-dibenzylimidazo[1,2-a]pyridin-2(3H)-one,
3,3-dibenzyl-8-cyclopentyloxyimidazo[1,2-a]pyridin-2(3H)-one,
3,3-dibenzyl-6,8-dichloroimidazo[1,2-a]pyridin-2(3H)-one,
3,3-dibenzyl-8-chloro-6-trifluoromethylimidazo[1,2-a]pyridin-2(3H)-one,
8-benzyloxy-3,3-bis(3-methylbenzyl)imidazo[1,2-a]pyridin-2(3H)-one,
8-methyl-3,3-bis(4-pyridylmethyl)imidazo[1,2-a]pyridin-2(3H)-one,
3,3-bis(4-fluorobenzyl)imidazo[1,2-a]pyridin-2(3H)-one,
3,3-bis(4-dimethylaminobenzyl)imidazo[1,2-a]pyridin-2(3H)-one,
3,3-bis(3-chlorobenzyl)imidazo[1,2-a]pyridin-2(3H)-one,
3,3-bis(4-methoxybenzyl)imidazo[1,2-a]pyridin-2(3H)-one,
3,3-bis(4-biphenylmethyl)imidazo[1,2-a]pyridin-2(3H)-one,
3,3-bis(4-cyanobenzyl)imidazo[1,2-a]pyridin-2(3H)-one,
3,3-bis(4-hydroxybenzyl)imidazo[1,2-a]pyridin-2(3H)-one,
3,3-diallylimidazo[1,2-a]pyridin-2(3H)-one,
3,3-diallyl-8-benzyloxyimidazo[1,2-a]pyridin-2(3H)-one,
3,3-bis(3-phenyl-1-propyl)imidazo[1,2-a]pyridin-2(3H)-one, spiro[imidazo[1,2-a]pyridin-2(3H)-one-3,2′-[2,3]dihydrophenalene],
3,3-bis(2,4-difluorobenzyl)imidazo[1,2-a]pyridin-2(3H)-one,
3,3-dipropylimidazo[1,2-a]pyridin-2(3H)-one,
3,3-bis(2-thienylmethyl)imidazo[1,2-a]pyridin-2(3H)-one,
8-acetylamino-3,3-dibenzylimidazo[1,2-a]pyridin-2(3H)-one,
3,3-bis(2-furylmethyl)imidazo[1,2-a]pyridin-2(3H)-one,
3,3-dimethylimidazo[1,2-a]pyridin-2(3H)-one,
3,3-dibutylimidazo[1,2-a]pyridin-2(3H)-one,
3,3-di(2-propinyl)imidazo[1,2-a]pyridin-2(3H)-one,
3,3-dibenzyl-8-hydroxyimidazo[1,2-a]pyridin-2(3H)-one,
3,3-dibenzyl-8-benzylaminoimidazo[1,2-a]pyridin-2(3H)-one,
3,3-bis(4-nitrobenzyl)imidazo[1,2-a]pyridin-2(3H)-one,
8-amino-3,3-dibenzylimidazo[1,2-a]pyridin-2(3H)-one,
3,3-bis(4-methoxycarbonylbenzyl)imidazo[1,2-a]pyridin-2(3H)-one,
5,5-bis(4-fluorobenzyl)imidazo[2,1-b]thiazol-6(5H)-one,
5,5-dibenzylimidazo[2,1-b]thiazol-6(5H)-one,
3,3-dibenzylimidazo[1,2-a]pyrimidin-2(3H)-one,
5,5-bis(4-methylbenzyl)imidazo[2,1-b]thiazol-6(5H)-one,
5,5-bis(4-cyanobenzyl)imidazo[2,1-b]thiazol-6(5H)-one,
5,5-dibenzyl-2-methylimidazo[2,1-b]thiazol-6(5H)-one,
5,5-bis(2-thienylmethyl)imidazo[2,1-b]thiazol-6(5H)-one,
3,3-bis(2-thienylmethyl)imidazo[1,2-a]pyrimidin-2(3H)-one,
5,5-dibenzyl-2,3-dihydroimidazo[2,1-b]thiazol-6(5H)-one,
2-hydroxy-3-(2-naphthylmethyl)imidazo[1,2-a]pyridine,
spiro[imidazo[1,2-a]pyridin-2(3H)-one-3,2′-benzo[f]indan],
3-benzylimidazo[1,2-a]pyridin-2(3H)-one,
3,3-di(2-butenyl)imidazo[1,2-a]pyrimidin-2(3H)-one,
spiro[imidazo[1,2-a]pyridin-2(3H)-one-3,2′-(4′-fluoroindan)],
spiro[imidazo[1,2-a]pyridin-2(3H)-one-3,2′-(5′-methoxyindan)],
spiro[imidazo[1,2-a]pyridin-2(3H)-one-3,2′-(5′-iodoindan)],
spiro[imidazo[1,2-a]pyridin-2(3H)-one-3,2′(4′-cyanoindan)],
spiro[imidazo[2,1-a]isoquinolin-2(3H)-one-3,2′-indan],
spiro[imidazo[1,2-a]pyridin-2(3H)-one-3,2′-[1,2,5]thiadiazo[4,5-c]indan],
spiro[imidazo[2,1-a]isoquinolin-2(3H)-one-3,2′-[1,2,5]thiadiazo[4,5-c]indan],
spiro[imidazo[1,2-a]pyrimidin-2(3H)-one-3,2′-indan],
spiro[imidazo[1,2-a]pyridin-2(3H)-one-3,2′-(5′-trifluoromethylindan)],
spiro[imidazo[1,2-a]pyridin-2(3H)-one-3,2′-benzo[e]indan],
3,3-diallylimidazo[1,2-a]pyridin-2(3H)-one,
3,3-bis(2-cyclohexenyl)imidazo[1,2-a]pyridin-2(3H)-one,
3,3-diallylimidazo[2,1-a]isoquinolin-2(3H)-one,
spiro[imidazo[2,1-a]isoquinolin-2(3H)-one-3,4′-(1′-cyclopentene)],
spiro[8-benzyloxyimidazo[1,2-a]pyridin-2(3H)-one-3,4′-(1′-cyclopentene)],
3,3-dipropyl-5,6,7,8-tetrahydroimidazo[1,2-a]pyridin-2(3H)-one,
3,3-dicyclohexyl-5,6,7,8-tetrahydroimidazo[1,2-a]pyridin-2(3H)-one,
3,3-dibutyl-5,6,7,8-tetrahydroimidazo[1,2-a]pyridin-2(3H)-one,
spiro[7,8,9,10-tetrahydroimidazo[2,1-a]isoquinolin-2(3H)-one-3,1′-cyclopentane],
spiro[imidazo[2,1-a]isoquinolin-2(3H)-one-3,1′-cyclopentane],
spiro[5,6,7,8-tetrahydroimidazo[1,2-a]pyridin-2(3H)-one-3,2′-benzo[f]indan],
spiro[5,6,7,8-tetrahydroimidazo[1,2-a]pyridin-2(3H)-one-3,2′-indan],
3,3-bis(4-chlorobenzyl)imidazo[1,2-a]pyridin-2(3H)-one,
8-cyclopropylmethyloxy-3,3-diallylimidazo[1,2-a]pyridin-2(3H)-one,
spiro[imidazo[1,2-a]pyridin-2(3H)-one-3,2′-(4′-hydroxyindan)],
spiro[8-hydroxyimidazo[1,2-a]pyridin-2(3H)-one-3,2′-indan],
spiro[8-methoxyimidazo[1,2-a]pyridin-2(3H)-one-3,4′-(1′-cyclopentene)],
spiro[8-cyclopropylmethyloxyimidazo[1,2-a]pyridin-2(3H)-one-3,4′-(1′-cyclopentene)],
8-amino-3,3-dibenzylimidazo[1,2-a]pyridin-2(3H)-one hydrochloride,
8-benzylamino-3,3-dibenzylimidazo[1,2-a]pyridin-2(3H)-one, and spiro[8-acetylaminoimidazo[1,2-a]pyridin-2(3H)-one-3,2′-indan].

The heterocyclic compound is more preferably at least one heterocyclic compound chosen from the group consisting of:
3,3-dibenzylimidazo[1,2-a]pyridin-2(3H)-one,
spiro[imidazo[1,2-a]pyridin-2(3H)-one-3,2′-indan],
3,3-dipropylimidazo[1,2-a]pyridin-2(3H)-one,
3,3-dibutylimidazo[1,2-a]pyridin-2(31)-one,
5,5-dibenzylimidazo[2,1-b]thiazol-6(5H)-one,
3,3-dibenzylimidazo[1,2-a]pyrimidin-2(3H)-one,
spiro[imidazo[1,2-a]pyridin-2(3H)-one-3,2′-(4′-fluoroindan)],
spiro[imidazo[1,2-a]pyridin-2(3H)-one-3,2′-(5′-methoxyindan)],
spiro[imidazo[1,2-a]pyridin-2(3H)-one-3,2′-(4′-cyanoindan)],
spiro[imidazo[2,1-a]isoquinolin-2(3H)-one-3,2′-indan],
spiro[imidazo[1,2-a]pyridin-2(3H)-one-3,2′-[1,2,5]thiadiazo[4,5-c]indan],
spiro[imidazo[1,2-a]pyrimidin-2(3H)-one-3,2′-indan],
spiro[imidazo[2,1-a]isoquinolin-2(3H)-one-3,4′-(1′-cyclopentene)],
3,3-bis(4-chlorobenzyl)imidazo[1,2-a]pyridin-2(3H)-one,
8-cyclopropylmethyloxy-3,3-diallylimidazo[1,2-a]pyridin-2(3H)-one,
spiro[imidazo[1,2-a]pyridin-2(3H)-one-3,2′-(4′-hydroxyindan)],
spiro[8-hydroxy-imidazo[1,2-a]pyridin-2(3H)-one-3,2′-indan],
spiro[8-methoxyimidazo[1,2-a]pyridin-2(3H)-one-3,4′-(1′-cyclopentene)],
spiro[8-acetylaminoimidazo[1,2-a]pyridin-2(3H)-one-3,2′-indan]
   and
spiro[8-cyclopropylmethyloxyimidazo[1,2-a]pyridin-2(3H)-one-3,4′-(1′-cyclopentene)].

More preferably, the heterocyclic compound is spiro[imidazo[1,2-a]pyridin-2(3H)-one-3,2′-indan].

The cognitive impairment may be caused by cerebrovascular disease, Lewy body dementia, Alzheimer's disease, Parkinson's disease, Pick's disease, Huntington's disease or Down's syndrome, or may be memory impairment due to aging.

The therapeutic agent for neurodegenerative disease is preferably an acetylcholinesterase inhibitor, such as donepezil hydrochloride, rivastigmine tartrate or galantamine hydrobromide, or a non-competitive NMDA receptor antagonist such as memantine hydrochloride.

The therapeutic agent for neurodegenerative disease and the heterocyclic compound, hydrate thereof or pharmaceutically acceptable salt thereof may be administered simultaneously, separately or consecutively.

### BRIEF DESCRIPTION OF DRAWINGS

FIG. 1 depicts a graphical representation for explaining the activity of compound 1 (ZSET1446) and donepezil on cognitive impairment induced by scopolamine in a passive avoidance task in mice. Each value represents the mean S.E.M. The number within the column indicates the number of animals. ##P<0.01, compared with vehicle-treated control group (Mann-Whitney U-test). *P<0.05, **P<0.01, compared with scopolamine-treated rats given 1% CMC (Steel's test). ++P<0.01, compared with the group treated with 1% CMC+donepezil (0.01 mg/kg) and scopolamine (Steel's test). $ P<0.05, compared with the group treated with 1% CMC+donepezil (0.1 mg/kg) and scopolamine (Steel's test).

### DETAILED DESCRIPTION OF THE INVENTION

The embodiments of the present invention are described hereafter.

According to a first aspect of the present invention, a kit or medicament for treating cognitive impairment, includes a therapeutic agent for neurodegenerative disease and a heterocyclic compound represented by the following General Formula (I):

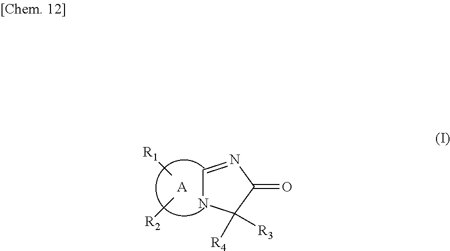

or a hydrate thereof, a solvate thereof or a pharmaceutically acceptable salt thereof.

According to a second aspect of the present invention, a pharmaceutical composition for treating cognitive impairment, includes a therapeutic agent for neurodegenerative disease and a heterocyclic compound represented by the following General Formula (I):

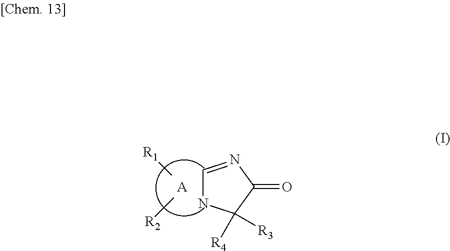

or a hydrate thereof, a solvate thereof or a pharmaceutically acceptable salt thereof.

According to a third aspect of the present invention, a composition includes a therapeutic agent for neurodegenerative disease and a heterocyclic compound represented by the following General Formula (I):

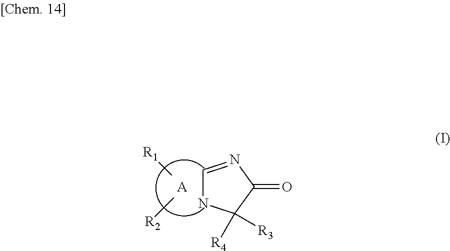

or a hydrate thereof, a solvate thereof or a pharmaceutically acceptable salt thereof.

According to a fourth aspect of the present invention, a product includes a therapeutic agent for neurodegenerative disease and a heterocyclic compound represented by the following General Formula (I):

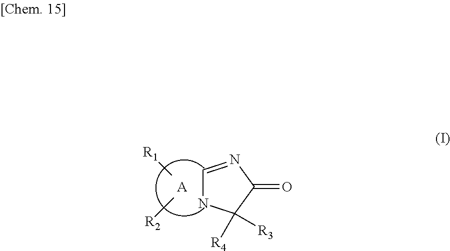

or a hydrate thereof, a solvate thereof or a pharmaceutically acceptable salt thereof, as a combined preparation for simultaneous, separate or sequential use in treatment of cognitive impairment.

According to a fifth aspect of the present invention, a medicament includes a therapeutic agent for neurodegenerative disease, for use in treatment of cognitive impairment in combination with a heterocyclic compound represented by the following General Formula (I):

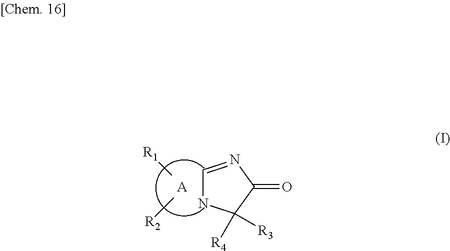

or a hydrate thereof, a solvate thereof or a pharmaceutically acceptable salt thereof.

According to a sixth aspect of the present invention, a medicament includes a heterocyclic compound represented by the following General Formula (I):

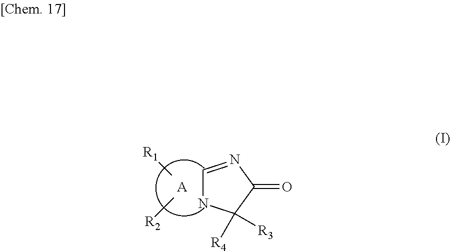

or a hydrate thereof, a solvate thereof or a pharmaceutically acceptable salt thereof, for use in treatment of cognitive impairment in combination with a therapeutic agent for neurodegenerative disease.

According to a seventh aspect of the present invention, a method of treating cognitive impairment, includes administering to a subject in need thereof a combination of a therapeutic agent for neurodegenerative disease and a heterocyclic compound represented by the following General Formula (I):

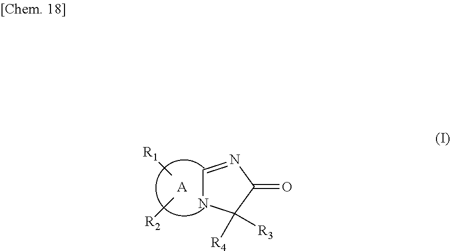

or a hydrate thereof, a solvate thereof or a pharmaceutically acceptable salt thereof.

In the General Formula (I), the structural unit having the General Formula (II):

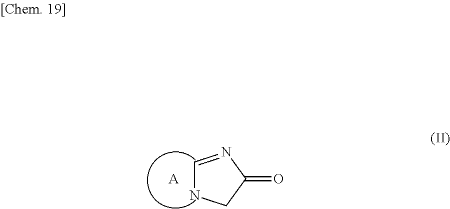

is selected from structural units having the General Formula

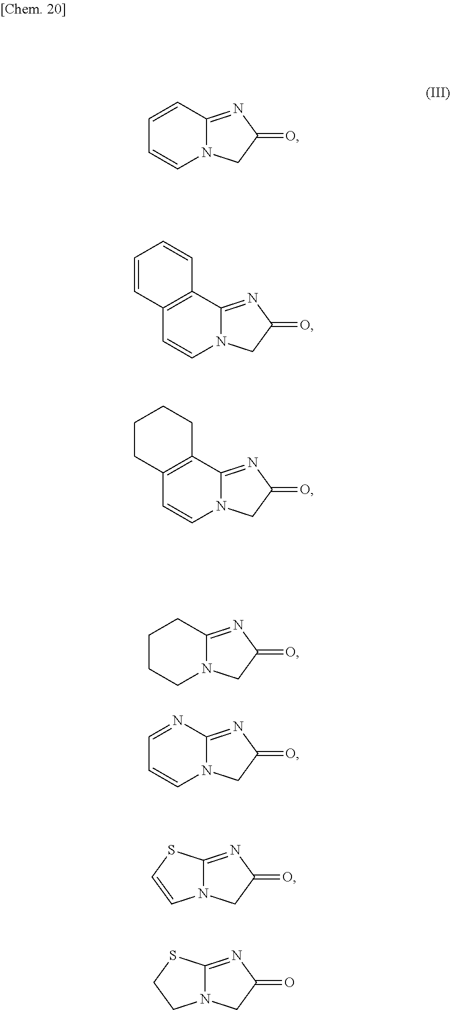

Furthermore, in the General Formula (I), R₁ and R₂ each are independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, and —O—(CH₂)ₙ—R₅, wherein R₅ is a vinyl group, C₃-C₆ cycloalkyl group, or phenyl group, and n is 0 or 1.

Furthermore, in the General Formula (I), R₃ and R₄ each are independently selected from the group consisting of a hydrogen atom, C₁-C₆ alkyl group, C₃-C₈ cycloalkyl group, and —CH(R₇)—R₆; alternatively, R₅ and R₄ together form a spiro ring having the General Formula (IV);

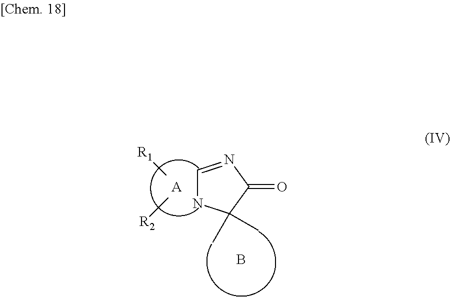

The above R₅ is selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group. The above R₇ is a hydrogen atom or C₁-C₆ alkyl group.

In the General Formula (IV), the structural unit B is selected from multiple types of structural units having the General Formula (V). The structural unit B binds at a position marked by * in the General Formula (V) to form a spiro ring:

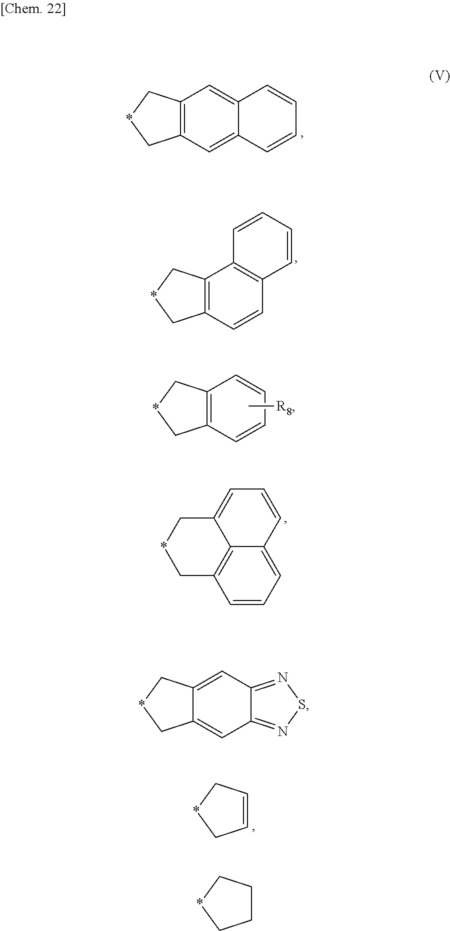

Here, R₈ is one or more substituent group(s) selected from the group consisting of a hydrogen atom, halogen atom, hydroxy group, C₁-C₆ alkoxy group, cyano group, and trifluoromethyl group.

When the heterocyclic compound having the General Formula (I) has asymmetric carbon atoms in the structure, its isomer from asymmetric carbon atoms and their mixture (racemic modification) is present. In such cases, all are included in the heterocyclic compound used in the embodiments described later.

The heterocyclic compound has the General Formula (I). In the General Formula (I), the following terms have the meanings specified below along with their examples.

The term “C₁-C₆” refers to 1 to 6 carbon atoms, unless otherwise defined. The term “C₃-C₈” refers to 3 to 8 carbon atoms, unless otherwise defined. The term “C₁-C₆ alkyl” includes linear or branched alkyl groups, such as methyl, ethyl, n-propyl, isopropyl, n-butyl, tert-butyl, sec-butyl, n-pentyl, and n-hexyl. The term “C₁-C₆ alkoxy” includes linear or branched alkoxy groups, such as methoxy, ethoxy, n-propoxy, isopropoxy, n-butoxy, tert-butoxy, sec-butoxy, n-pentyloxy, and n-hexyloxy. The term “C₃-C₈ cycloalkyl” includes cyclopropyl, cyclobutyl, cyclopentyl, cyclohexyl, cycloheptyl, and cyclooctyl. The term “halogen atom” includes fluorine, chlorine, bromine, and iodine.

The heterocyclic compound useful in the practice of the present invention is not particularly restricted as long as it has the above described specific structure. For example, the following compounds can be used:
spiro[imidazo[1,2-a]pyridin-2(3H)-one-3,2′-indan],
3,3-dibenzyl-8-isopropoxyimidazo[1,2-a]pyridin-2(3H)-one,
3,3-dibenzyl-8-methoxyimidazo[1,2-a]pyridin-2(3H)-one,
3,3-dibenzyl-8-cyclopropylmethyloxy-imidazo[1,2-a]pyridin-2(3H)-one,
3,3-dibenzyl-6-chloroimidazo[1,2-a]pyridin-2(3H)-one,
8-allkyloxy-3,3-dibenzylimidazo[1,2-a]pyridin-2(3H)-one,
3,3-dibenzyl-8-benzyloxyimidazo[1,2-a]pyridin-2(3H)-one,
8-benzyloxy-3,3-bis(1-phenylethyl)imidazo[1,2-a]pyridin-2(3H)-one,
3,3-dibenzyl-8-methylimidazo[1,2-a]pyridin-2(3H)-one,
3,3-dibenzyl-5,7-dimethylimidazo[1,2-a]pyridin-2(3H)-one,
3,3-dibenzylimidazo[1,2-a]pyridin-2(3H)-one,
3,3-dibenzyl-8-cyclopentyloxyimidazo[1,2-a]pyridin-2(3H)-one,
3,3-dibenzyl-6,8-dichloroimidazo[1,2-a]pyridin-2(3H)-one,
3,3-dibenzyl-8-chloro-6-trifluoromethylimidazo[1,2-a]pyridin-2(3H)-one,
8-benzyloxy-3,3-bis(3-methylbenzyl)imidazo[1,2-a]pyridin-2(3H)-one,
8-methyl-3,3-bis(4-pyridylmethyl)imidazo[1,2-a]pyridin-2(3H)-one,
3,3-bis(4-fluorobenzyl)imidazo[1,2-a]pyridin-2(3H)-one,
3,3-bis(4-dimethylaminobenzyl)imidazo[1,2-a]pyridin-2(3H)-one,
3,3-bis(3-chlorobenzyl)imidazo[1,2-a]pyridin-2(3H)-one,
3,3-bis(4-methoxybenzyl)imidazo[1,2-a]pyridin-2(3H)-one,
3,3-bis(4-biphenylmethyl)imidazo[1,2-a]pyridin-2(3H)-one,
3,3-bis(4-cyanobenzyl)imidazo[1,2-a]pyridin-2(3H)-one,
3,3-bis(4-hydroxybenzyl)imidazo[1,2-a]pyridin-2(3H)-one,
3,3-diallylimidazo[1,2-a]pyridin-2(3H)-one,
3,3-diallyl-8-benzyloxyimidazo[1,2-a]pyridin-2(3H)-one,
3,3-bis(3-phenyl-1-propyl)imidazo[1,2-a]pyridin-2(3H)-one,
spiro[imidazo[1,2-a]pyridin-2(3H)-one-3,2′-[2,3]dihydrophenalene],
3,3-bis(2,4-difluorobenzyl)imidazo[1,2-a]pyridin-2(3H)-one,
3,3-dipropylimidazo[1,2-a]pyridin-2(3H)-one,
3,3-bis(2-thienylmethyl)imidazo[1,2-a]pyridin-2(3H)-one,
8-acetylamino-3,3-dibenzylimidazo[1,2-a]pyridin-2(3H)-one,
3,3-bis(2-furylmethyl)imidazo[1,2-a]pyridin-2(3H)-one,
3,3-dimethylimidazo[1,2-a]pyridin-2(3H)-one,
3,3-dibutylimidazo[1,2-a]pyridin-2(3H)-one,
3,3-di(2-propynyl)imidazo[1,2-a]pyridin-2(3H)-one,
3,3-dibenzyl-8-hydroxyimidazo[1,2-a]pyridin-2(3H)-one,
3,3-dibenzyl-8-benzylaminoimidazo[1,2-a]pyridin-2(3H)-one,
3,3-bis(4-nitrobenzyl)imidazo[1,2-a]pyridin-2(3H)-one,
8-amino-3,3-dibenzylimidazo[1,2-a]pyridin-2(3H)-one,
3,3-bis(4-methoxycarbonylbenzyl)imidazo[1,2-a]pyridin-2(3H)-one,
5,5-bis(4-fluorobenzyl)imidazo[2,1-b]thiazol-6(5H)-one,
5-dibenzylimidazo[2,1-b]thiazol-6(5H)-one,
3,3-dibenzylimidazo[1,2-a]pyrimidin-2(3H)-one,
5,5-bis(4-methylbenzyl)imidazo[2,1-b]thiazol-6(5H)-one,
5,5-bis(4-cyanobenzyl)imidazo[2,1-b]thiazol-6(5H)-one,
5,5-dibenzyl-2-methylimidazo[2,1-b]thiazol-6(5H)-one,
5,5-bis(2-thienylmethyl)imidazo[2,1-b]thiazol-6(5H)-one,
3,3-bis(2-thienylmethyl)imidazo[1,2-a]pyrimidin-2(3H)-one,
5,5-dibenzyl-2,3-dihydroimidazo[2,1-b]thiazol-6(5H)-one,
2-hydroxy-3-(2-naphthylmethyl)imidazo[1,2-a]pyridine,
spiro[imidazo[1,2-a]pyridin-2(3H)-one-3,2′-benzo[f]indan],
3-benzylimidazo[1,2-a]pyridin-2(3H)-one,
3,3-di(2-butenyl)imidazo[1,2-a]pyrimidin-2(3H)-one,
spiro[imidazo[1,2-a]pyridin-2(3H)-one-3,2′-(4′-fluoroindan)],
spiro[imidazo[1,2-a]pyridin-2(3H)-one-3,2′-(5′-methoxyindan)],
spiro[imidazo[1,2-a]pyridin-2(3H)-one-3,2′-(5′-iodoindan)],
spiro[imidazo[1,2-a]pyridin-2(3H)-one-3,2′-(4′-cyanoindan)],
spiro[imidazo[2,1-a]isoquinolin-2(3H)-one-3,2′-indan],
spiro[imidazo[1,2-a]pyridin-2(3H)-one-3,2′-[1,2,5]thiadiazo[4,5-c]indan],
spiro[imidazo[2,1-a]isoquinolin-2(3H)-one-3,2′-[1,2,5]thiadiazo[4,5-c]indan],
spiro[imidazo[1,2-a]pyrimidin-2(3H)-one-3,2′-indan],
spiro[imidazo[1,2-a]pyridin-2(3H)-one-3,2′-(5′-trifluoromethylindan)],
spiro[imidazo[1,2-a]pyridin-2(3H)-one-3,2′-benzo[e]indan],
3,3-diallylimidazo[1,2-a]pyridin-2(3H)-one,
3,3-bis(2-cyclohexenyl)imidazo[1,2-a]pyridin-2(3H)-one,
3,3-diallylimidazo[2,1-a]isoquinolin-2(3H)-one,
spiro[imidazo[2,1-a]isoquinolin-2(3H)-one-3,4′-(1′-cyclopentene)],
spiro[8-benzyloxyimidazo[1,2-a]pyridin-2(3H)-one-3,4′-(1′-cyclopentene)],
3,3-dipropyl-5,6,7,8-tetrahydroimidazo[1,2-a]pyridin-2(3H)-one,
3,3-dicyclohexyl-5,6,7,8-tetrahydroimidazo[1,2-a]pyridin-2(3H)-one,
3,3-dibutyl-5,6,7,8-tetrahydroimidazo[1,2-a]pyridin-2(3H)-one,
spiro[7,8,9,10-tetrahydroimidazo[2,1-a]isoquinolin-2(3H)-one-3,1′-cyclopentane],
spiro[imidazo[2,1-a]isoquinolin-2(31-1)-one-3,1′-cyclopentane],
spiro[5,6,7,8-tetrahydroimidazo[1,2-a]pyridin-2(3H)-one-3,2′-benzo[f]indan],
spiro[5,6,7,8-tetrahydroimidazo[1,2-a]pyridin-2(3H)-one-3,2′-indan],
3,3-bis(4-chlorobenzyl)imidazo[1,2-a]pyridin-2(3H)-one,
8-cyclopropylmethyloxy-3,3-diallylimidazo[1,2-a]pyridin-2(3H)-one,
spiro[imidazo pyridin-2(3H)-one-3,2′-(4′-hydroxyindan)],
spiro[8-hydroxyimidazo[1,2-a]pyridin-2(3H)-one-3,2′-indan],
spiro[8-methoxyimidazo[1,2-a]pyridin-2(3H)-one-3,4′-(1′-cyclopentene)],
spiro[8-cyclopropylmethyloxyimidazo[1,2-a]pyridin-2(3H)-one-3,4′-(1′-cyclopentene)],
8-amino-3,3-dibenzylimidazo[1,2-a]pyridin-2(3H)-one hydrochloride,
8-benzylamino-3,3-dibenzylimidazo[1,2-a]pyridin-2(3H)-one, or
spiro[8-acetylaminoimidazo[1,2-a]pyridin-2(3H)-one-3,2′-indan].

The heterocyclic compound of Formula (I) can be in the form of hydrate, solvates or acid addition salts as a pharmaceutically acceptable salt. Possible solvates include organic solvates, such as dimethylsulfoxide solvate, N,N-dimethylformamide solvate or alcohol solvates like ethanol, methanol and n-propanol solvates. Possible acid addition salts include inorganic acid salts, such as hydrochloride, sulfate, hydrobromide, nitrate, and phosphate salts or organic acid salts, such as acetate, oxalate, propionate, glycolate, lactate, pyruvate, malonate, succinate, maleate, fumarate, malate, tartrate, citrate, benzoate, cinnamate, methanesulfonate, benzenesulfonate, p-toluenesulfonate, and salicylate salts.

The therapeutic agent for neurodegenerative disease used in the present invention is not particularly restricted, but should preferably be one or more drugs chosen from among the acetylcholinesterase inhibitors, donepezil hydrochloride, rivastigmine tartrate and galantamine hydrobromide; and the non-competitive NMDA receptor antagonist, memantine hydrochloride.

The method of treatment of the present invention or the method of treatment using the present invention is by a drug regimen combining (A) a heterocyclic compound indicated by the above General Formula (I), a hydrate thereof, a solvate thereof or a pharmaceutically acceptable salt thereof, and (B) a therapeutic agent for neurodegenerative disease. Additionally, drug A and drug B may themselves be combinations of a plurality of drugs, and may also contain auxiliary drugs, diluents and carriers. The treatment method of the present invention may be by combining drug A and drug B into the same pharmaceutical composition, or by administering drug A and drug B simultaneously, separately or consecutively. Additionally, if to be administered separately, drug A may be administered before drug B, or conversely, drug B may be administered before drug A. The method of delivery and the number of doses per day may be the same or different, and there is no particular limitation on the weight ratio between drug A and drug B.

Moreover, at the time of storage, transport, or commercial sale, a kit or product may be provided with drug A and drug B simultaneously stored therein. A practical kit or product may also be provided, in which drug A and drug B are separately prepared at the time of usage. Furthermore, drug A and drug B may each be provided with instructions or directions, in which the concomitant administration thereof is described.

The cognitive impairment may be caused by cerebrovascular disease, Lewy body dementia, Alzheimer's disease, Parkinson's disease, Pick's disease, Huntington's disease or Down's syndrome, or may be memory impairment due to aging.

Additionally, while the dosages of the heterocyclic compound indicated by the above General Formula (I), hydrate, solvate or pharmaceutically acceptable salt thereof, and the therapeutic agent for neurodegenerative disease according to the present embodiment will differ depending on age, weight, symptoms, therapeutic effects and method of delivery, the dosages should be at least about 0.0001 mg per kilogram of body weight in the case of oral delivery. More preferably, the content or dosage of the heterocyclic compound indicated by the above General Formula (I) should be at least about 0.001 mg/kg, and the dosage of the simultaneously used therapeutic agent for neurodegenerative disease should be at least about 0.01 mg/kg. Additionally, in another embodiment, these drugs may be delivered in units of 5, 10, 15, 20, 25, 30, 35, 40, 45, 50, 55, 60, 65, 70, 75, 80, 85, 90, 95 or 100 mg.

Additionally, in the case of oral delivery of a single preparation containing a heterocyclic compound indicated by the above General Formula (I), hydrate, solvate or pharmaceutically acceptable salt thereof, and a therapeutic agent for a neurodegenerative disorder, it can be offered in the form of an ingestible solid or an ingestible liquid for oral delivery.

Formulations for oral administration include ingestible solids, tablets, coated tablets, powders, granules, capsules, microcapsules and syrups.

These formulations or compositions can be prepared by using pharmacologically acceptable excipients, binders, lubricants, disintegrators, suspensions, emulsifiers, preservatives, stabilizers and dispersants such as lactose, sucrose, starch, dextrin, crystalline cellulose, kaolin, calcium carbonate, talc, magnesium stearate, distilled water and physiological saline solution.

The present inventors studied the effects, for example, of simultaneous administration of spiro[imidazo[1,2-a]pyridin-2(3H)-one-3,2′-indan] among the heterocyclic compounds indicated by the above General Formula (I) and together with donepezil hydrochloride as an acetylcholinesterase inhibitor on scopolamine-induced memory impairment in mice. As a result, they observed clear concomitant activity at dosages in which activity was not observed when the respective drugs were used alone.

Thus, low doses of the heterocyclic compounds indicated by the General Formula (I) and low doses of the therapeutic agents for treating a neurodegenerative disease may be coadministered. Consequently, regardless of whether these drugs only demonstrate limited effectiveness at lower doses, or whether these drugs demonstrate any conventional effects at all when coadministered in low doses, it is still possible that the above-mentioned drugs may be capable of inducing therapeutic activity, or may be capable of achieving superior therapeutic activity at lower doses. Such low doses are generally sub-therapeutic doses when the two agents are administered alone. Examples of such low doses include doses of less than 0.1 mg/kg of donepezil and less than 0.001 mg/kg of spiro[imidazo[1,2-a]pyridin-2(3H)-one-3,2′-indan], specifically, less than 0.01 mg/kg of donepezil and less than 0.0001 mg/kg of spiro[imidazo[1,2-a]pyridin-2(3H)-one-3,2′-indan]. With respect to human dosing, exemplary low doses include 1 mg, 2 mg, 3 mg or 4 mg donepezil hydrochloride and 1 mg, 2 mg, 3 mg, 4 mg or 5 mg of spiro[imidazo[1,2-a]pyridin-2(3H)-one-3,2′-indan]. The heterocyclic compounds indicated by the General Formula (I), e.g. spiro[imidazo[1,2-a]pyridin-2(3H)-one-3,2′-indan], and the therapeutic agents for treating a neurodegenerative disease, e.g. donepezil hydrochloride, may be administered as part of a single unitary pharmaceutical composition or may be part of separate pharmaceutical compositions.

Moreover, the heterocyclic compounds indicated by the General Formula (I) may be coadministered with effective doses of the therapeutic agents for treating a neurodegenerative disease. At such a time, the heterocyclic compounds indicated by the General Formula (I) may be administered in either low doses, or effective doses. Moreover, it is possible that when above-mentioned drugs are coadministered rather than individually administered, the therapeutic effects of the therapeutic agent for treating a neurodegenerative disease, or the therapeutic effects of the heterocyclic compounds indicated by the General Formula (I) are improved significantly. Examples of such effective doses include a dose of 0.1 mg/kg of donepezil and a dose of 0.01 mg/kg of spiro[imidazo[1,2-a]pyridin-2(3H)-one-3,2′-indan]. Examples of such low doses include a dose of less than 0.001 mg/kg of spiro[imidazo[1,2-a]pyridin-2(3H)-one-3,2′-indan], specifically, less than 0.0001 mg/kg of spiro[imidazo[1,2-a]pyridin-2(3H)-one-3,2′-indan]. With respect to human dosing, exemplary effective doses include 1 mg or 5 mg donepezil hydrochloride and 0.1 mg of spiro[imidazo[1,2-a]pyridin-2(3H)-one-3,2′-indan]. Exemplary low doses include 1 mg, 2 mg, 3 mg or 4 mg donepezil hydrochloride and 1 mg, 2 mg, 3 mg, 4 mg or 5 mg of spiro[imidazo[1,2-a]pyridin-2(3H)-one-3,2′-indan]. The heterocyclic compounds indicated by the General Formula (I), e.g. spiro[imidazo[1,2-a]pyridin-2(3H)-one-3,2′-indan], and the therapeutic agents for treating a neurodegenerative disease, e.g. donepezil hydrochloride, may be administered as part of a single unitary pharmaceutical composition or may be part of separate pharmaceutical compositions.

Moreover, when referring to the “effective amount” or “therapeutically effective amount”, not only does it refer to an amount that is individually effective in a treatment, but it also includes a subtherapeutic effective amount, which is an amount that is effective in combination with the present invention rather than alone.

Additionally, they studied the effect, for example, of simultaneous administration of spiro[imidazo[1,2-a]pyridin-2(3H)-one-3,2′-indan] among the heterocyclic compounds indicated by the above General Formula (I) and together with donepezil hydrochloride as an acetylcholinesterase inhibitor on the amount of extracellular acetylcholine in the hippocampus. As a result, a significant increase in the amount of extracellular acetylcholine was observed at a dosage in which activity was not observed with donepezil hydrochloride alone.

While preferred embodiments of the present invention have been described and illustrated above, it is to be understood that they are exemplary of the invention and are not to be considered to be limiting.

For example, some preferable ranges of effective oral dosages are defined in the above embodiments. However, other ranges of effective dosages can be determined for other administration forms. For example, a preferable range of effective dosages for administration can be determined as appropriate. Furthermore, preferable ranges of administration intervals can be determined for particular administration forms in addition to the effective dosages with no more than routine experimentation.

### EXAMPLES

Hereinafter, the present invention will be explained in greater detail with reference to the Examples. However, the present invention is not specifically limited to the below-mentioned Examples.

### Example 1

Combination effect of Compound 1 (spiro[imidazo-[1,2-a]pyridin-2(3H)-one-3,2′-indan]) and donepezil on scopolamine-induced cognitive impairment examined by passive avoidance tasks in mice.

### Methods

### Animals

Eight to nine week old male ICR strain mice (Charles River Laboratories Japan, Inc.) were used in the experiment. They were housed in a cage in groups of 3 or 4 mice, in a room maintained at around 22 degree C. under 12 hour light/dark cycles. Food and water were provided ad libitum. All animal care and treatment was conducted in accordance with the Guidelines for the Care and Use of Laboratory Animals, established at the Central Research Laboratory, Zenyaku Kogyo Co., Ltd.

### Drugs

Compound 1 and donepezil were suspended in 1% carboxymethyl cellulose (CMC). Scopolamine (Sigma) was dissolved in 0.9% NaCl. For the coadministration studies of Compound 1 and donepezil, both drug suspensions were mixed together and this mixed suspension was injected. All drugs were prepared immediately before use and orally administered at a dosage of 10 ml/kg.

### Passive Avoidance Task

The passive avoidance apparatus (Neuroscience Inc.) consisted of an illuminated chamber and a larger dark chamber. Two chambers were separated by a guillotine door. Oral administration of compound 1 at doses of 0.0001 mg/kg or 0.001 mg/kg and/or donepezil at doses of 0.01 and 0.1 mg/kg was given 60 min before the acquisition trial. Scopolamine (1 mg/kg) was intraperitoneally injected 20 min before the acquisition trial. The matched control group only received the vehicle. During the acquisition trial, each mouse was placed in the illuminated chamber. Immediately after the entry into the dark chamber, the door was closed and an inescapable scrambled electric shock (100 V, 0.4 mA, 1.5 sec) was delivered through the floor grid. Twenty-four hours later, each mouse was placed in the illuminated chamber for retention trial. The interval between placement in the illuminated chamber and entry into the dark chamber was measured as step through latency (maximum 300 seconds).

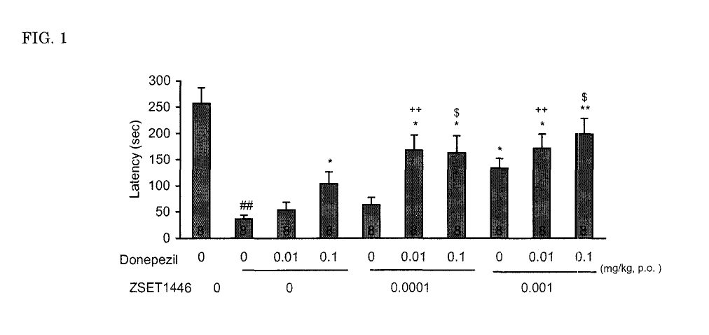

The results were compared between the 1% CMC-scopolamine group and 1% CMC-physiological saline groups using the Mann-Whitney U-test (shown as the second bar and the first bar, respectively, in FIG. 1). When there was a significant difference, the cognitive impairment was considered to be scopolamine induced. The results were compared to the 1% CMC-scopolamine group using Steel's test. A P level of <0.05 was considered indicative of statistical significance for the tests. Next, the results were compared to the 1% CMC+scopolamine group and the respective groups indicated by * and ** in FIG. 1; 1% CMC+donepezil (0.01 mg/kg)+scopolamine and compound 1 (0.0001 or 0.001 mg/kg)+donepezil (0.01 mg/kg)+scopolamine (indicated by ++ in FIG. 1); and 1% CMC+donepezil (0.1 mg/kg)+scopolamine and compound 1 (0.0001 or 0.001 mg/kg)+donepezil (0.1 mg/kg)+scopolamine (indicated by $ in FIG. 1), using Steel's test.

### Results

In the retention trial, the step-through latency in a group treated with 1% CMC and scopolamine was markedly shorter than that in the group treated with 1% CMC and saline (P<0.01). These results demonstrate that scopolamine impaired passive avoidance performance. Oral administration of compound 1 at a dose of 0.0001 mg/kg or donepezil at a dose of 0.01 mg/kg did not significantly prolong the step-through latency as compared with that in the group treated with 1% CMC and scopolamine. On the other hand, oral administration of donepezil at a dose of 0.1 mg/kg or compound 1 at a dose of 0.001 mg/kg prolonged the step-through latency (P<0.05).

Concomitant administration of compound 1 (0.0001 mg/kg), donepezil (0.01 or 0.1 mg/kg) and scopolamine significantly prolonged the step-through latency as compared with that in the group treated with 1% CMC and scopolamine (P<0.05). Moreover, concomitant administration of compound 1 (0.001 mg/kg), donepezil (0.1 mg/kg) and scopolamine significantly prolonged the step-through latency as compared with that in the group treated with 1% CMC and scopolamine (P<0.01). Moreover, concomitant administration of compound 1 (0.0001 or 0.001 mg/kg), donepezil (0.01 mg/kg) and scopolamine significantly prolonged the step-through latency as compared with that in the group treated with donepezil (0.01 mg/kg) and scopolamine (P<0.01). Similarly, concomitant administration of compound 1 (0.001 mg/kg), donepezil (0.1 mg/kg) and scopolamine also significantly prolonged the step-through latency as compared with that in the group treated with donepezil (0.1 mg/kg) and scopolamine (P<0.01).

The most important finding of the present study was that concomitant administration of compound 1 and donepezil at subeffective doses, as well as, at effective doses synergistically ameliorated cognitive impairment induced by scopolamine in the passive avoidance task. These results suggest the synergistic interaction of different mechanisms of the two drugs.

### Example 2

Combination effects of compound 1 and donepezil on extracellular levels of acetylcholine (ACh) in a rat hippocampus.

### Methods

### Animals

Eight to nine week old male Wistar strain rats (Japan Laboratory Animals Inc.) were used in the experiment. The rats were housed in a cage in groups of 2 or 3 rats, in a room maintained at around 22 C under 12 hour light/dark cycles. Food and water were provided ad libitum. All animal care and treatment was conducted in accordance with the Guidelines for the Care and Use of Laboratory Animals, established at the Central Research Laboratory, Zenyaku Kogyo Co., Ltd.

### Drugs

Compound 1 and donepezil were suspended in 1% CMC. For the coadministration studies of compound 1 and donepezil, both drug suspensions were mixed together and this mixed suspension was prepared immediately before use and orally administered at a dose of 1 ml/kg.

### Surgery

The rats were anesthetized with pentobarbital (50 mg/kg) and fixed in a stereotaxic apparatus (David Kopf Instruments, Tujunga, Calif., USA). The skull was exposed and a stainless-steel guide cannula (AG-8, Eicom, Kyoto) was implanted into the hippocampus (A-5.8; L 4.8; V 4.0 mm) according to the atlas of Paxinos and Watson (1982). The day after the operation, microdialysis probes with 3-mm-long cellulose membrane tubings (A-I-8-03, Eicom) were inserted into the hippocampus through the implanted guide cannula.

### ACh Measurement

The probes were perfused with Ringer's solution (147 mM NaCl, 4.02 mM KCl, and 2.25 n-LM CaCl₂) at a flow rate of 1.0 microlitter/min. Dialysates were collected every 20 min and the ACh level was detected by an HPLC system with electrochemical detection (ECD). ACh was separated from the dialysates using a column (Eicompac AC-Gel 2.0×150 mm, Eicom). The enzymatic reactor contained acetylcholinesterase (AC hE) and choline oxidase, which catalyzes the formation of hydrogen peroxide from ACh and choline. The resultant H₂O₂ was detected by ECD (ECD-300, Eicom), with a platinum electrode (WE-PT, Eicom) at 450 mV.

### Statistical Analysis

The statistical significance of the differences between groups was calculated by one-way analysis of variance, which was followed by Dunnett's multiple comparison test.

### Results

Oral administration of compound 1 at a dose of 0.001 mg/kg or donepezil at a dose of 1 mg/kg did not significantly increase the extracellular level of ACh in the hippocampus as compared with that in the group treated with 1% CMC. However, concomitant administration of compound 1 (0.001 mg/kg) and donepezil (1 mg/kg) significantly increased the extracellular level of ACh as compared with that in the group treated with 1% CMC.

The most important finding of the present study was that concomitant administration of compound 1 and donepezil at subeffective doses for each drug synergistically increased the extracellular level of ACh in the hippocampus.

### Preparation of Compounds Referred to in the Embodiments

Some of the heterocyclic compound having the General Formula (I) and prepared by the method(s) described in the examples of WO 01/09131 are described hereafter by way of example. More specifically, they were synthesized with reference to WO 01/09131 and WO 2002/060907 Brochure.

### Preparation

An exemplary preparation of spiro[imidazo[1,2-a]pyridin-2(3H)-one-3,2′-indan] (Compound 1) having the General Formula below is described hereafter. Compound 1:

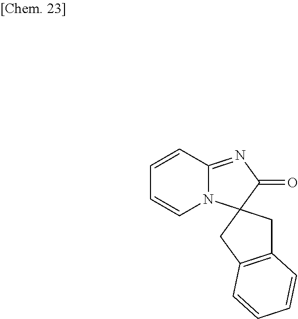

An amount of 56.1 g (1.04 mol) of sodium methoxide was dissolved in 15 L of methanol, and 90.0 g (0.0345 mol) of 2-amino-1-(ethoxycarbonylmethyl)pyridinium bromide and 60.0 g (0.0342 mol) of alpha,alpha'-dichloro-o-xylene were added successively at room temperature. The reaction mixture was stirred at room temperature over-night and then the solvent was removed under reduced pressure. Dichloromethane was added to the residue and insoluble matter was filtered off. The filtrate was concentrated under reduced pressure and the residue was passed through a silica gel column (ethyl acetate methanol=15:1) to yield a crude product. The crude product was washed using ethyl acetate and then recrystallized from methanol to provide 36 g (40%) of the title compound in the form of white crystals. The results of the analysis of the obtained compound are given below. The results show that the obtained compound was the targeted compound

Melting Point: 206 degree C. (decomposition);

NMR (CDCl₃) delta: 3.16 (2H, d, J=16 Hz), 3.89 (2H, d, J=16 Hz), 6.49 (1H, t, J=7 Hz), 7.1-7.2 (2H, m), 7.2-7.3 (4H, m), 7.61 (1H, t, J=7 Hz);

MS m/z: 236 (M⁺).

Other compounds of Formula (I) can be prepared from appropriate starting materials in a suitable manner according to WO 01/09131 and WO 02/060907, which are incorporated herein by reference.

The present invention is described above using examples. The examples are given by way of example. It is understood by a person in the art that various modifications are available and those modifications are included in the scope of the present invention.

For example, the above examples have used Compound 1 as the heterocyclic compound, dopenezil as the therapeutic agent for neurodegenerative disease, and mice as the mammalian subject thereof. However, other heterocyclic compounds, other therapeutic agents for neurodegenerative disease, and/or other mammals, including humans, can be used. The above compounds will also exhibit a therapeutic effect with regard to cognitive impairment in other mammals, including humans.

The disclosures of the patents, patent applications and publications cited in the present specification are hereby incorporated into the present specification by reference.

1. A kit comprising spiro[imidazo[1,2-a]pyridin-2(3H)-one-3,2′-indan] and a therapeutic agent selected from the group consisting of donepezil hydrochloride, rivastigmine tartrate, galantamine hydrobromide, and memantine hydrochloride.

2. The kit of claim 1, wherein the therapeutic agent is in a form suitable for oral administration.

3. The kit of claim 1, wherein the therapeutic agent is in the form of a tablet.

4. The kit of claim 1, wherein the therapeutic agent is donepezil hydrochloride.

5. The kit of claim 1, wherein the therapeutic agent is rivastigmine tartrate.

6. The kit of claim 1, wherein the therapeutic agent is galantamine hydrobromide.

7. The kit of claim 1, wherein the therapeutic agent is memantine hydrochloride.

8. A method of treating cognitive impairment in a subject in need thereof, comprising administering to the subject a therapeutically effective amount of spiro[imidazo[1,2-a]pyridin-2(3H)-one-3,2′-indan] and a therapeutic agent selected from the group consisting of donepezil hydrochloride, rivastigmine tartrate, galantamine hydrobromide, and memantine hydrochloride.

9. The method of claim 8, wherein the administration of said therapeutic agent and spiro[imidazo[1,2-a]pyridin-2(3H)-one-3,2′-indan] occurs simultaneously.

10. The method of claim 8, wherein the administration of said therapeutic agent and spiro[imidazo[1,2-a]pyridin-2(3H)-one-3,2′-indan] occurs separately.

11. The method of claim 8, wherein spiro[imidazo[1,2-a]pyridin-2(3H)-one-3,2′-indan], said therapeutic agent or a combination thereof are administered orally.

12. The method of claim 8, wherein the therapeutic agent is donepezil hydrochloride.

13. The method of claim 8, wherein the therapeutic agent is rivastigmine tartrate.

14. The method of claim 8, wherein the therapeutic agent is galantamine hydrobromide.

15. The method of claim 8, wherein the therapeutic agent is memantine hydrochloride.

16. The method of claim 8, wherein the subject is a human subject.

17. The method of claim 8, wherein the cognitive impairment is caused by Alzheimer's disease.

18. A composition comprising spiro[imidazo[1,2-a]pyridin-2(3H)-one-3,2′-indan] and a therapeutic agent selected from the group consisting of donepezil hydrochloride, rivastigmine tartrate, galantamine hydrobromide, and memantine hydrochloride

19. The composition of claim 18, which is in a form suitable for oral administration.

20. The composition of claim 18, wherein the therapeutic agent is donepezil hydrochloride.
